(19) United States
(12) Patent Application Publication
(10) Pub. No.: US 2010/0154681 A1
(43) Pub. Date: Jun. 24, 2010

(54) METHOD FOR MANUFACTURING BIOMEDICAL BONE FILLER WITH CONCRETE CHARACTERISTIC

(76) Inventor: Nan-Hui Yeh, Kaohsiung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/719,494

(22) Filed: Mar. 8, 2010

Related U.S. Application Data
(63) Continuation-in-part of application No. 11/653,217, filed on Jan. 16, 2007, now abandoned.

(30) Foreign Application Priority Data
Jan. 17, 2006 (CN) 200610001701.2

(51) Int. Cl. A61L 24/02; C04B 11/00

(52) U.S. Cl. 106/782

(57) ABSTRACT

A method for manufacturing a biomedical bone filler includes the steps of: mixing different size of granule and slag of hemihydrate calcium sulfate with particles of hemihydrate calcium sulfate at a predetermined particle ratio and powders/water ratio; and hardening the composite material by controlling relative humidity and temperature during the hydrated hardening process so as to increase the hardness of the bone filler.

This application is a CIP (Continuation In Part) of the application Ser. No. 11/653,217; titling “Method For Manufacturing Biomedical Bone Material With Concrete Characteristic”, filed on Jan. 16, 2007.

### BACKGROUND OF THE INVENTION

1. Field of the Invention

The present invention relates to a method for manufacturing biomedical bone filler, more particularly to a method for manufacturing biomedical bone filler having concrete characteristic feature.

2. Description of the Prior Art

In earlier days, the biomedical material is not applied in clinical treatment. It is applied in clinical treatment only after the sterilized surgical technology has been developed in 1860. Without considering the use of biomedical material in clinical treatment, the surgical operation done in those earlier years seldom bring fruit due to infection, which, in turn, prevents implant of the biomedical material into the human body and due to body's immunity. However, it is noted that calcium sulfate has nice biocompatibility, easily obtainable, cheap in expense and is adapted to be bound to the host bone. Thus, there is a history of hundred years that calcium sulfate (generally known as Plaster of Paris) is used in the clinical treatment, like orthopedic surgery, to stimulate growth of bone defects and serves as bone filler.

In recent years, calcium sulfate is used in the bone graft transplant to fill the voids due to bone fracture and delayed union of bone tissue in the bone fracture, osteoma and osteomyelitis, thereby preventing growth of soft fiber therein. In the past, two bone graft methods are available depending on the initial source of bone graft, namely: (i) autologous bone graft method and (ii) allograft method. In the autologous bone graft method, the transplant bone graft generally comes from the skeletal system of the patient. As a rule, the bone graft can be extracted from the patient is limited. In the allograft method, the transplant bone graft comes from the other persons, animals or Bone and Tissue Bank (special refrigerator). In practice, this method brings some disadvantages; like one cannot be sure whether the bone grafts are infected or not, one cannot rest assure whether the bone graft is free from virus or diseases and response to immunity. In recent years with the advent of modern medicine on skeletal components, the scientists are doing researches to develop new bone substitute (filler), hoping to fill the voids caused in the human skeleton or due to bone infection. The new bone filler should possess clinical benefits, like autologus intracavernous bone graft, to fill the position of the difference in order to produce the desired shape. The new bone filler should also possess post-implanted compatibility with the blood vessel and ultimately assists in growth of normal bone tissue. In post-implant time, the bone filler should provide adequate strength and support to the bone structure, completely free infection, being cheap one can input an appropriate amount of antibiotics so as not only can prevent infection, and also some growth factor can be added in order to enhance reconstruction of the bone structure.

The skeletal system supports the skull, two legs and two hands, hence the body, and protects the inner organs. In case, the human body is impacted, the skeletal system may be injured, thereby causing discomforts in our daily life. A great devotion is to replace a partial of human organ with a biomedical filler. The skeletal system may unfortunately have some disadvantages due to disease, like tumor, which requires to be removed by surgery. Although, the bone tissue, compared to and were lower than in other organs, have excellent self-restoration ability, but when bone defect is too large, the osteocyte themselves are unable to repair fast. Under this condition, an appropriate bone filler is required to be implanted in order to fill the voids and provides temporary mechanical strength for to support the defected bone and shorten the clinical treating time.

Regarding the bone graft, in autologous transplant, the transplant bone graft generally comes from the skeletal system of the patient. As a rule, this method provides the outmost biocompatibility and bone tissue repair ability, safety and effective result to the patient. The bone graft can be extracted from the patient is limited and the person undergoing the autologous transplant is often left with a surgical mark or scar, thereby causing the patient psychologically unsecure and mentally discomfort. Therefore, it is relatively difficult to use autologous transplant for bone grafting to replace the major defected bone part. In the allograft transplant, the transplant bone graft comes from different persons. In practice, this method brings sufficient quantity to repair the bone defects but one cannot be sure whether the donated person of the bone graft has immunological response, thereby leading to failure in transplant operation. There is yet another type called xenograft, wherein the bone graft comes from different living species such that the quantity is not limited but there may be complicated transplant due to different species of immunological response. Therefore, scientists are eager to develop a biomedical bone filler having different-species biocompatibility and low degradation such that the patients can be free from being infected due to implant of the bone graft. Calcium sulfate and calcium phosphate respectively are ceramic material having nice biocompatibility and thus serve as major component in manufacturing of the presently used bone filler.

Note that calcium sulfate, possesses osteoconductive ability so that it is used to fill the voids in the defected bone part, since it enhances restoration the profile of the bone structure and prevents infiltration of soft fibers into the voids. Calcium sulfate can be bound to blood vessels and osteocytes and cooperatively provide osteoconductive ability such that the biomedical bone filler can be absorbed swiftly in the bone part to restores its initial shape.

Calcium sulfate (Plaster) is a naturally found mineral, its chemical compound is CaSO₄.2H₂O, can be obtained in two ways: (i) naturally and (ii) chemically. Chemically produced plaster includes FGD (flue gas desulfurization) and residue material. For FGD, in order to prevent pollution caused by the electricity power mill, the plaster is mixed together with water to form slurry of cement to absorb SO₂ within the smoke, thereby forming Ca(HSO₃)₂, after oxidation becomes dihydrate calcium sulfate (CaSO₄.2H₂O). The following table shows how Plaster of Paris is produced after reaction among chemicals.

| | |
|---|---|
| Resorption | 2SO₂ + H₂O + CaCO₃ → Ca(HSO₃)₂ + CO₂ |
| neutralize | CaCO₃ + H₂SO₄ → CaSO₄ + CO₂ + H₂O |
| Oxidation | Ca(HSO₃)₂ + O₂ →CaSO₄ + H₂SO₄ |
| Crystallization | CaSO₄ + 2H₂O →CaSO₄□2H₂O |

For production of hemihydrate calcium sulfate (CaSO₄.1/2H₂O), the dihydrate calcium sulfate (CaSO₄.2H₂O) is passed through a compressed steam treatment of 120˜150° C. to obtain α-CaSO₄.1/2H₂O. In case of passing through a dried atmosphere with 110˜130° C., β-CaSO₄.1/2H₂O is achieved, wherein these two types have different grain configuration, the α-CaSO₄.1/2H₂O is composed of a plurality of granule with small total surface area and large diameter while β-CaSO₄.1/2H₂O is composed of a plurality of slag with large total surface area and small diameter. After thermogravimetric analysis test, it is found that α-CaSO₄.1/2H₂O possesses a tiny peak of exothermic reactions after absorption of heat, but the β-CaSO₄.1/2H₂O does not.

During the hydrated hardening process of hemi-hydrate calcium sulfate (CaSO₄.1/2H₂O), CaSO₄.1/2H₂O dissolves in water to form Ca²⁺ and SO₄²⁻, gradually becomes hydrated material having crystals of dihydrate calcium sulfate (CaSO₄.2H₂O). Under the same temperature and during the hydrated hardening process, the β-CaSO₄.1/2H₂O possesses greater exothermic ability with shorter time when compared to the α-CaSO₄.1/2H₂O. Throughout the course of hydrated hardening process, due to different types of the crystals, the β-CaSO₄.1/2H₂O owing to large surface area requires more amount of water since slag with small diameter dissolves quickly in the water, thus shortening the hydration process.

The main function of hard tissue is to support and protect the soft tissue in the human body. In case the hard tissue is accidentally injured or fatigued due to disease or aging, it is hard to be repaired and may cause the injured person physically disabled and inconvenience in moving about. Therefore, the medical researchers are devoting their greatest afford to develop an ideal biomedical bone substitute having fine osteoinductive ability to induce osteoinductive growth to assist in forming new bone growth, hence the osteoconductive framework. There are six types of artificial bone substitute materials.

(1) Autografts: the bone substitute is fetched from skeletal bone or bone marrow of the patient, is the best in view of biological compatibility. It is generally fetched from the patient's pelvic and has high fusion rate to benefit the patient. There is no risk of tissue rejection, bone infection and pain. However, some patients with smoking habits or over obesity may produce failed fusion after implant.

(2) Allografts and Allograft-based: the bone substitute is fetched from other living or dead body, and is used together with other biocompatible material. Its advantages are optimum structural support; easy for shaping, demineralised bone to help growth in patient's bone tissue. Its disadvantage is that in case of using the bone substitute lonely, there is limited growth in patient's bone tissue and it may transmit infection or disease.

(3) Ceramic-based: Calcium phosphate, calcium sulfate, glass are used lonely or in combination with other material. The advantage is that the bone graft substitute thus manufactured is cheap in cost. The disadvantage is that, if ceramic-based material becomes a majority part of the bone graft substitute, the latter is susceptible to fragile when supporting heavy load.

(4) Polymer-based: Resoluable or non-resoluable polymer is used lonely or in combination with other material. The advantage is that the bone graft substitute thus manufactured has high absorption and provides adequate strength. The disadvantage is that it has non osteoinductive ability to induce osteoinductive growth of bone tissue.

(5) Factor-based: the artificial bone material combined with growth hormone mainly includes chemicals and proteins for controlling the cell activity. Presently the main research is focused on study of bone morphogenetic protein (Proteins). Some of the factors have proved the growth of osteoinductivity and is implanted via carrier frame in clinical trials. The cost to manufacture this type of bone substitute is high.

(6) Cell-based: since mesenchymal stem cells in human body are undifferentiated cells, a specific chemical must be added to induce cell development into osteoblast. In animal experiments, the implanted containing human mesenchymal stem cells and bone marrow and porous ceramic material. The same way can be applied in cartilage or tendon repair and regeneration thereof. The benefits are creating many different types of tissue and becoming bioactive material.

In earlier times, artificial materials used for tissue repair mainly includes 316 L stainless steel, cobalt-chromium alloys, titanium alloy, corrosion-resistant metallic materials and the PMMA silicone resin, high density Polyethylene, etc. Therefore, the artificial bone substitutes are targeted to biocompatible and bioinductive materials, like calcium sulfate and calcium phosphate, to assist in restoration of bone structure. The following table shows mechanical strength of natural bone parts.

| | Modulus of | Tensile | Compressive |
|---|---|---|---|
| | elasticity | strength | strength |
| | (GPa) | (MPa) | (MPa) |
| Femur | 17.2 | 121 | 167 |
| Tibia | 18.1 | 140 | 159 |
| Fibula | 18.6 | 146 | 123 |
| Humures | 17.2 | 130 | 132 |
| Radius | 18.6 | 149 | 114 |
| Ulna | 18.0 | 148 | 117 |

The following table shows mechanical strength of natural teeth.

| | Modulus of | Tensile | Compressive |
|---|---|---|---|
| | elasticity | strength | strength |
| | (GPa) | (MPa) | (MPa) |
| Enamel | 48 | 10-70 | 241 |
| Dentin | 13.8 | 50-60 | 138 |

Biomedical ceramics is divided into 3 categories depending on responsive action among the animal tissue.

(1) Completely Resorbable Bioceramics

Resoluble bio-ceramic material includes composition quite close to hard tissue in the human body and are absorbable in body fluid to result in chemical reaction, like solution-mediated processes such that activation of osteoclasts can continually remold the implant material so as to be replaced by osteoids eventually. Due to cell-mediated processes, the bio-ceramic material dissolves and the implanted material consequently minimizes in dimension. If the dissolving and tissue growth rates are controlled appropriately, the bone particles will replace the bio-ceramic material gradually and finally. This structure provides strength and response rate higher than the other two ceramic materials.

Paris (Plaster of Paris) includes inorganic materials with high resorption rate. In animal experiments, it dissolves more quickly than natural bone graft and provides mild tissue response in post-implant. Shortcomings are the absorption rate changes greatly and is low mechanical strength, thus limiting the application. In recent years, the Paris is mixed with hydrogen and oxygen-based hydroxyapatite and is implanted into a cat's head. The result is quite good one.

Calcium phosphate has excellent resorption ability. Calcium phosphate (CP), TCP (tri-calcium phosphate), four calcium phosphate (TECP), and the hydrogen and oxygen-based hydroxyapatite (HAp) have compressive strength of about 30 MPa and are preferred for supporting minor load.

(2) Biomedical Inert Ceramics

This material can stay stably in human fluid for long periods, releases no ion or tissue response. If there is a reaction, a very thin layer of fibrous form is formed on the implanted ceramic surface. On the other hand, one can machine on the inert ceramic surface to form holes so as to increase the organ's contact area, causing and enhancing mechanical adhesion thereof.

Biological inert ceramics (nearly inert ceramics) has developed since the 1960s, a considerable amount of material was developed ever since. Represent material, such as silicon dioxide, alumina, zirconia, and so on. Due to its highly biocompatible and stability, high structural strength, high hardness and wear-resistant, the scope of application in human organisms is rather wide. The main application is at the artificial joints, serving grinding socket surface. Presently, several products are developed due to its high hardness and wear-resistant and can stay long in human body, and can reduce friction wear as encountered in the conventional metal product and issues of metal ions.

(3) Surface Reactive Ceramics

This material can establish chemical bond with the surrounding tissues. Since the responsive action is only on the external surface, and does not affect the strength of the original material. The material can also be coated on the other material surface, such as stainless steel, alloys, aluminum oxide, etc Co—Cr, so that it is a reactive surface. The main composition is hydrogen and oxygen-based phosphate and hydroxyapatite (glass).

The use of surface reactive ceramics in orthopedic clinical trails is restricted owing to its fragility. A separate application is not ready to stress at the site of most application can not bear the stress so that it acts as a filler to fill in repair of bone voids, or as a coating on the metal surface so that the metal substitute can combine strongly on the bone parts.

Surface reactive ceramics in body fluid environment, due to the degradation of the material or dissolving-release action, have pH value change so that the calcium phosphate ions are released from the material surface, resulting in a layer of Silicon (Si-rich), the dissolution of calcium phosphate ion being highly concentrated at a certain portion to form apatite crystals in the direction along the crystallization reprecipitation. Due to regeneration along the contact surface of the bone, hydroxyapatite on the surface will link collagen fibers on the bone defect, after which, collagen fibrous grow and are mineralized and forming solid structure.

In 1969, Hench and associates have discovered that bioglass (Na₂O—CaO—SiO₂—P₂O₅) is directly related to skeletal system. After implanted into human body, the bioglass dissolves into Na⁺ ion, forming a layer of collagen fiber-rich SiO₂. Finally, the bioglass dissolves and releases Ca⁺² ion and P⁺⁵ ion, forming crystallization of hydroxyapatite in the vicinity of collagen fibrous and is bound to the bone part. In 1973, a new Na₂O—K₂O—MgO—CaO—SiO₂—P₂O₅ system is developed, and named, Ceravital, and is produced by melting and cooling processes so as to partially form apatite crystal layer. The glass ceramics consisting apatite though has high mechanical strength is low bioactivity on its surface. Bioglass is one type of glass-ceramics (Glass-Ceramics), is referred to a solid crystal material containing a relative amount of glass. It is manufactured by glass melting and then molding and (controlled crystallization) heat treatment, making it crystal formation of solid polycrystalline. Recently, CaF₂ and TiO₂ are added to allow process easier, and addition of MgO does not affect the biological activity.

The bioglass has good affinity, due to its strength problem, is limited in the use. Clinically, its used is limited only to replacement of the middle ear bones and the Alveolar Ridge reconstruction. At present, the more successful instrument ERMI (Endosseous Ridge Maintenance Impllant), its components are Na₂O—CaO—SiO₂—P₂O₅, is used in dental implants. In recent years, bioglass with more strength of up to 200 MPa has been developed, its crystal composition includes Apatite and Wollastonite, also known as A-W glass.

Glass-ceramic is also known as sintered glass. This kind of material has good compression strength, stability in chemical action, and contains calcium and phosphorus compounds similar to the natural bone. More importantly, it has excellent biocompatibility.

Glass-ceramic contains a solid crystal material containing a relative amount of glass. It is manufactured by glass melting and then molding and (controlled crystallization) heat treatment, making it crystal formation of solid polycrystalline. Its feature is no porous structure at the inner portion, and has better properties of ceramic, physiological characteristic of different environments, and have biocompatible ability. For clinical used, the glass-ceramics should satisfy the requirements at different environmental and physical features of the application, and must have a bio-compatibility (biocompatible), biological activity (bioactive) and bounding ability between bony and cartilaginous tissue. It is often used together with hydrogen and oxygen-based hydroxyapatite (hydroxyapatite, HA), dicalcium phosphate (dicalcium phosphates: DCP) and tricalcium phosphate (TCP). These materials, owing to lack of sufficient mechanical strength, are limited in practice in many ways. However these materials are easy to form shape, has good bio-compatibility and so on, so that is used for reconstruction of hard-tissue, in particularly in orthopedics and dental replacement.

The features of composition of glass-ceramics are as follow.

(i) Less than 60% of SiO2;

(ii) Composition of Na₂O and CaO is as high as 30%

(iii) High ratio of CaO/P₂O₅

The following table shows the application and characteristic of bioglass and glass-ceramics.

| name | Bioglass | Ceravital | Glass-ceramics |
|---|---|---|---|
| Composition | Na₂O—CaO—P₂O₅—SiO₂ | Na₂O—K₂O—MgO—CaO—P₂O₅—SiO₂ | Na₂O—K₂O—MgO—CaO—Al₂O₃—P₂O₅—SiO₂—F |
| Phase | Glass | Apatite | Apatite |
| crystallization | | | Gold-mica |
| Fold | 85 | 150 | 140-220 |
| resistance | | | |
| Compression | NA | 500 | 550 |
| strength | | | |
| Flexibility | 79 | NA | 77-88 |
| (MPa) | | | |
| K_{IC} | 0.5 | NA | 0.5-1 |
| Bio-compatibity | Good | Good | Good |
| Extent of | Low | Intermediate | Intermediate |
| reconstruction | | | |
| Machining | Easy | Easy | Easy |
| Usage | Ear bone | dental | Artificial |
| | Coating | replacement | teeth root |
| | over | Slurry of bone | |
| | metal | cement | |

| | | New |
|---|---|---|
| | A-W | biomedical |
| name | Glass-ceramics | Glass-ceramics |
| Composition | MgO—CaO—P₂O₅—SiO₂ | Na₂O—CaO—P₂O₅—SiO₂ |
| Phase crystallization | Apatite | Na₂Ca₃Si₆O₁₆ |
| | Wollastonite | β-Ca₂P₂O₇ |
| Fold resistance | 220 | 120-140 |
| Compression strength | 1100 | 600-750 |
| Flexibility (MPa) | 120 | NA |
| K_{IC} | 2.0 | NA |
| Bio-compatibity | Good | Good |
| Extent of reconstruction | High | High |
| Machining | Easy | Easy |
| Usage | Artificial | Artificial |
| | teeth root | Bone |
| | Artificial | |
| | Bone | |

Calcium sulfate (gypsum) implanted in the human body to fill bone defect can be traced as earliest as 1892, where Dreesmann first mixes plaster slurry (Slurry of Paris) with 5% phenol and implants to fill bone defects in 8 patients. Through the tracking and found that six patients had bone growth into the plaster. Hence people start researching bone filler materials ever since.

In 1925, Kofmann implanted calcium sulfate to fill the voids in bone defects caused due to osteomyelitis. In 1952, Hauptli reported that he implanted calcium sulfate to fill the voids in bone defects in 16 patients and found it is safe and effective. Kovacevic used plaster consisting of penicillin and sulfonamide to the void due to osteomyelitis and removal of osteo stem. During the Vietnam War, calcium sulfate is used to repair facial bone parts. As for calcium phosphate, Jarcho conducted research in 1977 on relation between calcium phosphate and growth of bone tissue in the skeletal system. In 1985, Harvey implanted calcium phosphate under the skin of a rabbit. In 1988, Holmes implanted porous hydroxylapatite into the maxilla of a dog to find out variation of the tissue history. Rawlings implanted CaSO₄+(Ca₃(PO₄)₂) into a cat skull to observe the variation in bone tissue structure and found that after implantation of the porous hydroxyapatite, the growth of bone tissue is quickened.

In recent three years, three types of filler materials are put under experiment.

(1). OsteoSet; CaSO₄—H₂O is heat treated to become CaSO₄-1/2H₂O, which is passed through Peltier's experiment and proves to possess biocompatible property without increasing the environmental imflammation. Siduietal has discovered that osteoblasts can directly contact the implants while osteoclasts actively absorb calcium sulfate to form the normal bone iaculae. At the same time, the formation of acidic environment (pH5.6) during dissolution of the calcium sulfate can inhibit the activity of bacteria. Animal experiments have also confirmed that the more the bone graft implant absorb and its mechanical strength after implantation are similar to allograft which has gone frozen process.

(2) Norian Skeletal Repair System (SRS) uses a biocompatible and a resolvable slurry of calcium phosphate, which is formed by mixing together monocalcium phosphate, tricalcium phosphate, calcium carbonated and sodium phosphate solution. When this filler material is used in animal experiment, the osteoclasts resorb and form bone tissue after biomineralization process.

(3) ETEX α-BSM is a bone slurry formed by mixing dicalcium phosphatedihydrate, octocalcium phosphate and apatites. The composition formula is simulated from bone mineralization structure and is dried out for 20 minutes under the normal temperatures, its crystal arrangement is similar to natural bone and it helps bone resorption into their own original bone tissue. The most unique advantage is that one can add the antibiotics to promote the growth of bone tissue protein (growth factor).

As for human skeleton, the composition is a perfect composite materials including a large number of cytoplasm cells, intracellular Matrix and cells scattered among the cytoplasm cells. The cytoplasm is mainly composed of matrix, inorganic salts and water. The matrix is a network structure of organic matter, including 90%˜95% of the collagen, 1% of glycosaminoglycan and 5% of other protein. The inorganic salts mainly include calcium-deficient hydroxyapatite. The cytoplasm cells not only provide calcium, phosphorus and other materials necessary for metabolism within the skeletal system, but also mechanical strength for the major bones after calcification.

The physiological and chemical characteristics of the skeleton system are that the mature bone includes 45% of water, 25% of carbon powder and 20% of protein and 10% fat main constituents are: (1) bone mineralization; (2) collagen fiber; (3) shapeless fatty acids; and (4) water.

Collagen fibers are formed due to metabolism of osteoblasts, ⅓ of its amino acid composition is nucleotide, ⅓ preserved fruit acid and ⅓ other amino acid. Bone mineralization includes Ca, P, (OH), CO₃ and other salt ions. The crystallization salt mostly includes Ca₁₀(PO₄)₆—(OH)₂, each crystal has about 25-75 Å width, 200-600 Å length and are appeared on or inside the collagen fibers in compact state. In addition, collagen fibers section overlaps with one another so as the bone crystals, this structure is similar to the steel and concrete slurry. The collagen fiber structure is similar to steel rib while bone structure is similar to concrete. Thus, the skeletal system can withstand vibration forces and pressures. The proportion of fresh to bone, for compact bone, is about 1.9; and their, resistance to pressure and tension levels are 130 and 100 MPa respectively, therefore tougher than a steel wire.

The hard bone is a specialized structure containing cell calcification material, bone matrix and three different types of cells. At this point, let's discuss the following three types of cells: namely, osteoblasts, osteocytes and osteoclasts.

Osteoblast is formed from mesenchymal cells, that is, the distal end-cells can no longer crack. There may be two ways, firstly; formation of osteoprogenitor cell, secondly; induction of osteogenic cell. The decisive evolution is related with cell condensation, Concentrates forms in the embryo period and had direction related with osteocyte forms. Decisive evolution and cell formation are directly related to generation of the embryo osteocyte (embryogenesis). During the reconstruction and patching process, the osteoblast is susceptible to induce the effects of gene chemonucleolysis.

When a person head is injured due to accident or the surgery, a large number of cells at the wounded part is in osseous form amd function. Partial cells are obtained from the initial bone species, the rest including periosteum, endosteum and dura can induce the osteogenic cell. For example, this type of cell pericytes will take 3-5 days to destroy the wound, it is also possible within this cell membranes due to the growth of BMPs to convert into the osteocytes. In the earlier 12 hours, various forms of cytoplasm cells gather around the wound, for serving as initial source for formation of osteoblasts so as to repair blastema, during which mesenchymal stem cells in the bone marrow supply a complete cell. Because these cells due to being in different environment, such as nutrition, special growth factors, blood vessels and mechanical stability, provide a different potential conversion to form chondrocytes or osteoblasts (osteogenetic cells?).

Osteoblasts is a kind of cells dissolved due to metabolism of biological activity of secretory cells, (such as BMPs, TGF-β, insulin growth factor-I and II, tabular growth factor (PDGF)). During the reconstruction and repairing process, osteoblasts represent these products for femoral germ formation, for instance in the redevelopment process, osteoid is produced at 2-3 μm speed each day while osteogenetic cell is mineralized at 1 to 2 μm speed under the influence of osteoblasts.

Osteocytes are generally covered by cytoplasm? and stay within the caves formed at the adjoining area between bone plates. Osteocyte itself is a relatively inactive cells, however it plays a major role in controlling living cell activity on bone survival and the inner body balance. The complicated balance is conducted via controlling and adjusting physiological facts of the cells, tissue structure and inner organs such as growth facts.

Osteoclast cell is a multi-kernel cell formed by fusing a number of monocytes. The difference between osteoclast cell and huge osteoclast cell lies in that the osetoclasts have folded peripheral calcitonin receivers at the boundary for manufacturing tartaric acid and phosphate salt. The osetoclasts under this form can serve as a medium for bone resorption in physiological role. Osteoblasts and osetoclasts are dedicated to dynamic absorption of contact and interaction. For responding the secondary thyroid hormone PTH, osteoblasts are distributed over the outer surface of the bone, the mineralization of osteoid provides an opportunity to contact osetoclasts. This contact provides molecular and protein adsorption, and the folding boundary absorbs osteoclasts due to damaged bone surfaces. The area in contact with folding boundary and bone between the surface of the micro-environment, makes the enzyme to join the surrounding environment so as to drop the environment pH drop therefore increasing calcium and phosphate soluble in non-organic materials.

In the past, it is assumed that osteoclasts comes from osteoblasts, and they can also be sprouted from multi-kernel cells and then changes back to stem cells to form a mononuclear osteocyte. However, this hypothesis in the recent evidence proves that osetoclasts are achieved from fusion of mononuclear in blood.

FIG. 1 shows the reconstruction circle or steps of bone cell. The reconstruction steps includes osteoclasts activation, resorption and formation. In the osteoclasts activation, the monocytes are differentiated and proliterated, are latter fused into multi-kernel cells via the growth factors such as IL-1, ODP, finally become the activated osteoblasts. The activated osteoblasts starts bone resorption. The osteoblasts includes 5 to 50 monocytes or even more, and are dissolved in body fluid to form organic salt substances, which adhere to the defected bone parts for rehabilitation, thereby carrying out bone resorption. At the start of bone remodeling, the phagocyte gathers at the bony surface and is fused into osteoclasts, and then start absorbing OSTEOPORSIS. After three weeks of activationor so, a little tunnel of diameter 1 mm is formed for forming osteoblasts (bone osteoblast) at the same time. During the new bone formation, osteoclast cells will disappear, which is called apoptosis, and then osteoblasts will be attracted to the defected parts for rehabilitation. The osteoblasts are first of all form into bone matrix, and after mineralization a new bone is molded, wherein 10-20% of osteogenetic cells remain in the newborn bone as mineralized osteocytes.

### SUMMARY OF THE INVENTION

The main object of the present invention is to provide a method for manufacturing biomedical hemi-hydrate calcium sulfate tablet by controlling the relative humidity. The hemi-hydrate calcium sulfate tablet thus obtained is cracked into and is graded into plurality granule and slag, and is then mixed with particles of hemi-hydrate calcium sulfate at a predetermined powders ratio to form bone, void filler with concrete characteristic. After carrying out E. I. S. (electrochemical impedance spectrum), solidification time (setting time) for hardening the bone void filler can be calculated.

The other object of the present invention is to provide heating process, where steam generated due to heating the pure water and where the hemihydrate calcium sulfate is dried out via the circulation fan to increase the hardness of the hemihydrate calcium sulfate tablet, thereby forming a moist hemihydrate calcium sulfate tablet having an exterior portion consisting of dihydrate calcium sulfate crystals phase and an interior portion consisting of hemihydrate calcium sulfate crystals phase. The moist tablet is then dried out within a vacuum environment via the vacuum suction to obtain a compact hemihydrate calcium sulfate tablet.

In accordance of the present invention, each granule obtained by cracking the compact hemihydrate calcium sulfate tablet has a diameter ranging 200˜1500 μm while each slag has a diameter smaller than 50˜200 μm.

The above-mentioned granules and slag are graded via a standard sieve and are mixed together with a predetermined particles ratio of hemihydrate calcium sulfate powders to a composite material.

The composite material is later mixed with a solution to form a slurry of bone cement.

During the hydrated hardening process of the slurry of bone cement, the latter is measured at preset time interval to check the compression strength thereof.

The measured compression strength is compared to a blank compression strength, wherein the blank compression strength is measured after solidification of the bone cement at the optimum water ratio.

The optimum water ratio is adjusted when the measured compression strength is not compatible with the blank compression strength until the measured compression strength is compatible with the blank compression strength.

The method of manufacturing biomedical bone void filler of the present invention includes the steps of:

(a) preparing one biomedical hemihydrate calcium sulfate tablet;

(b) keeping said hemihydrate calcium sulfate tablet under a conditioning environment having constant temperature and humidity;

(c) controlling temperature of said conditioning environment at 40° C. with humidity ranging 50˜95% RH for 12 hours in order to form a moist hemihydrate calcium sulfate tablet, wherein said moist hemihydrate calcium sulfate tablet having an exterior portion consisting of dihydrate calcium sulfate crystal phase;

(d) drying out said moist hemihydrate calcium sulfate tablet under a vacuum condition in order to obtain a compact hemihydrate calcium sulfate tablet;

(e) cracking said compact hemihydrate calcium sulfate tablet into granules and slag;

(f) mixing particles of hemihydrate calcium sulfate with said granules and slag at a predetermined particles ratio and powers/water ratio to form a hemihydrate calcium sulfate composite material, which is mixed together with a solution to form a slurry of bone cement;

(g) carrying out a hardening process on said slurry of bone cement and measuring a compression strength of said slurry bone cement during said hardening process;

(h) comparing said measured compression strength relative to a blank compression strength, wherein said blank compression strength is measured after solidification of said bone cement at the optimum water ratio; and

(i) adjusting the optimum water ratio when said measured compression strength is not compatible with said blank compression strength until said measured compression strength is compatible with said blank compression strength.

### BRIEF DESCRIPTION OF THE DRAWINGS

Other features and advantages of this invention will become more apparent in the following detailed description of the preferred embodiments of this invention, with reference to the accompanying drawings, in which:

FIG. 1 shows the reconstruction circle or steps of bone cell;

FIGS. 2 and 2A are graphs representing a first test of the biomedical bone filler of the present invention to illustrate cracking time VS. weight under constant temperature and humidity;

FIGS. 3 and 3A are graphs representing a second test of the biomedical bone filler of the present invention to illustrate tracking time VS. weight under constant temperature and humidity;

FIG. 4 shows a cross section view of crystal structure of the hemihydrate calcium sulfate tablet manufactured according the method of the present invention prior to relative humidity treatment, wherein the cross section is magnified 3000 times under SEM (Scanning Electronic Microscope);

FIG. 5 shows a cross section view of crystal structure of the hemihydrate calcium sulfate tablet manufactured according the method of the present invention after relative humidity treatment, wherein the cross section is magnified 1500 times under SEM to illustrate an exterior portion;

FIG. 6 shows a cross section view of crystal structure of the hemihydrate calcium sulfate tablet manufactured according the method of the present invention after relative humidity treatment, wherein the cross section is magnified 1500 times under SEM to illustrate an interior portion;

FIG. 7 shows a cross section view of crystal structure of the hemihydrate calcium sulfate tablet manufactured according the method of the present invention after relative humidity treatment, wherein the cross section is magnified 1500 times under SEM to illustrate an intermediate portion;

FIG. 8 shows a graph of representing hardness test of the hemihydrate calcium sulfate tablet manufactured according the method of the present invention and is carried out by Vicker's Hardness Tester to show density table of the biomedical material after heat treatment for the first to fourth embodiments of the present invention;

FIG. 9 illustrates a distribution state of a plurality of slag in the biomedical bone filler of the present invention, viewed and magnified 50 times under SEM (Scanning Electronic Microscope);

FIG. 10 illustrates a distribution state of a plurality of granule in the biomedical bone filler of the present invention, viewed and magnified 50 times under SEM (Scanning Electronic Microscope);

FIG. 11 is a graph based on power and water ratio of column 1 after conducting E. I. S (electrochemical impedance spectrum) on the biomedical bone filler of the present invention;

FIG. 12 is a graph based on power and water ratio of column 2 after conducting E. I. S (electrochemical impedance spectrum) on the biomedical bone filler of the present invention;

FIG. 13 is a graph based on power and water ratio of column 3 after conducting E. I. S (electrochemical impedance spectrum) on the biomedical bone filler of the present invention;

FIG. 14 is a graph based on power and water ratio of column 4 after conducting E. I. S (electrochemical impedance spectrum) on the biomedical bone filler of the present invention;

FIG. 15 shows the crystals of hemihydrate calcium sulfate magnified 5000 times under SEM (Scanning Electronic Microscope) after solidification of the slurry of bone cement 1, 3, 5, 7, 9 and 11 minutes respectively during a solidification process in manufacturing the biomedical filler of the present invention;

FIG. 15 shows the crystals of hemihydrate calcium sulfate magnified 5000 times under SEM (Scanning Electronic Microscope) after solidification of the slurry of bone cement 1, 3, 5, 7, 9 and 11 minutes respectively; and

FIG. 16 shows the sixth embodiment of the biomedical bone filler of the present invention.

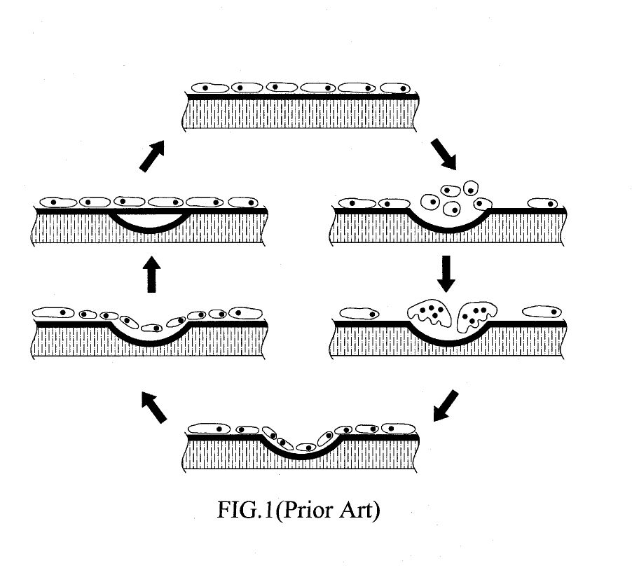

### DETAILED DESCRIPTION OF THE PREFERRED EMBODIMENTS

The method of the present invention is used for increasing the hardness of biomedical hemihydrate calcium sulfate table by controlling the relative humidity, thereby manufacturing a biomedical bone filler, which is used as bone graft for filling voids in the defected bone structure. Some instruments (such as a heater, a steam generator and circulation fan) are employed to let the steam passed through a vacuum room for increasing the hardness thereof and thus transforming into a moist hemihydrate calcium sulfate table. The moist hemihydrate calcium sulfate table has an exterior portion consisting of dihydrate calcium sulfate crystal phase and an interior portion consisting of hemihydrate calcium sulfate crystal phase.

In the first embodiment, a hemihydrate calcium sulfate table of 3 mm×5 mm, weight 0.115±0.05 g is prepared and is passed through a heat treatment under the constant temperature of 40° C. for 12 hours with different humidity ranging 50% RH, 65% RH, 75% RH, 85% RH and 95% RH. After thus treated, the hemihydrate calcium sulfate table is dried out (40° C. for 1 hours) in two different ways (i) via vacuum drying process and (ii) without the vacuum drying process. A test of cracking relative to hardness of the hemihydrate calcium sulfate table is conducted according to US medical standard.

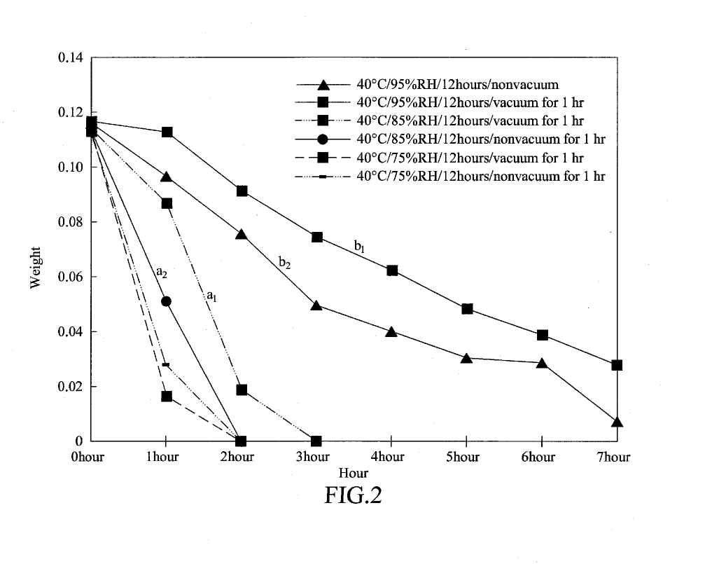

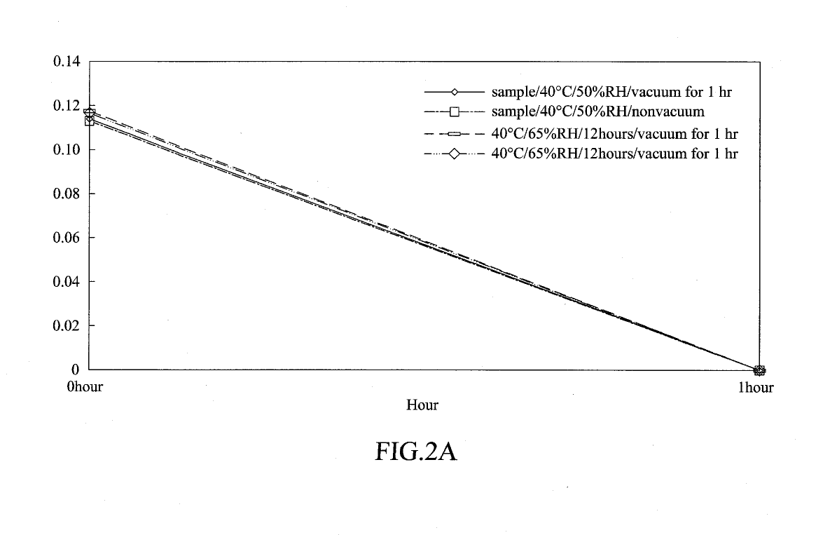

As illustrated in FIG. 2, wherein line a1 denotes that the tablet is heated for 12 hours under the constant temperature of 40° C. with relative humidity 85% RH and the environment is vacuumed for 1 hour while line a2 denotes that the tablet is heated for 12 hours under the constant temperature of 40° C. with relative humidity 85% RH and the environment is not vacuumed for 1 hour. Line b1 denotes that the tablet is heated for 12 hours under the constant temperature of 40° C. with relative humidity 95% RH and the environment is vacuumed for 1 hour I while line b2 denotes that the tablet is heated for 12 hours under the constant temperature of 40° C. with relative humidity 95% RH and the environment is not vacuumed for 1 hour.

After conducting a cracking test for the tablet, it is found that the cracking characteristic is evident under the constant temperature, increase of the cracking characteristic of the tablet is directly proportional to increase in the relative humidity of the tablet. In other words, by controlling the relative humidity of the tablet, one can let the tablet to absorb a relative humidity so as to prolong a required time for cracking the tablet. Moreover, the tablet that has undergone 1 hour of vacuum suction by controlling the relative humidity takes longer time to crack when compared to the table that has not gone 1 hour of vacuum suction. In short, the required time for cracking the tablet can be prolonged when the tablet is dried out via the vacuum suction process.

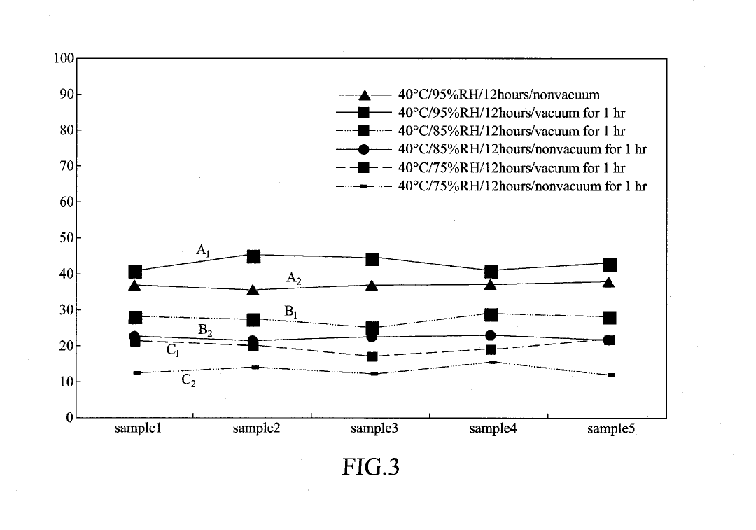

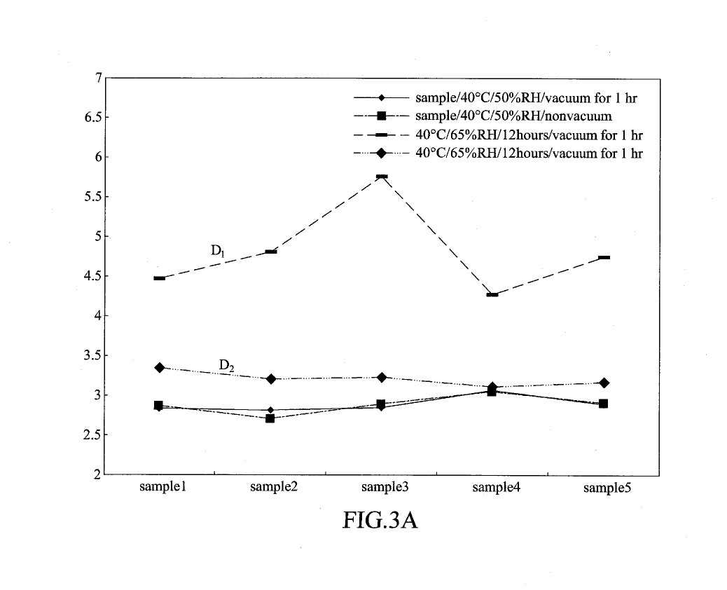

FIG. 3 shows the result after hardness test for the tablet. Line A 1 denotes that the tablet is passed through a heat treatment for 12 hours under the constant temperature of 40° C. with relative humidity 95% RH and the environment is vacuumed for 1 hour. Line A2 denotes that the tablet is passed through a heat treatment for 12 hours under the constant temperature of 40° C. with relative humidity 95% RH and the environment is not vacuumed for 1 hour. Line B1 denotes that the tablet is passed through a heat treatment for 12 hours under the constant temperature of 40° C. with relative humidity 85% RH and the environment is vacuumed for 1 hour while line B2 denotes that the tablet is passed through a heat treatment for 12 hours under the constant temperature of 40° C. with relative humidity 85% RH and the environment is not vacuumed for 1 hour. Line C1 denotes that the tablet is passed through a heat treatment for 12 hours under the constant temperature of 40° C. with relative humidity 75% RH and the environment is vacuumed for 1 hour while line C2 denotes that the tablet is passed through a heat treatment for 12 hours under the constant temperature of 40° C. with relative humidity 75% RH and the environment is not vacuumed for 1 hour. Line D1 denotes that the tablet is passed through a heat treatment for 12 hours under the constant temperature of 40° C. with relative humidity 65% R11 and the environment is vacuumed for 1 hour while line D2 denotes that the tablet is passed through a heat treatment for 12 hours under the constant temperature of 40° C. with relative humidity 65% RH and the environment is not vacuumed for 1 hour.

After conducting the hardness test for the tablet, it is found that under a constant temperature and time, the hardness of the tablet is increased as long as the relative humidity increases. In other words, by controlling the relative humidity, the hemihydrate calcium sulfate tablet can absorb more humidity in order to increase its hardness. Moreover, the tablet that has undergone 1 hour of vacuum suction by controlling the relative humidity has a greater hardness when compared to the table that has not gone 1 hour of vacuum suction. In short, the hardness of the tablet can be enhanced via the vacuum suction process. When a cross section of the tablet, which has undergone relative humidity treatment, is viewed under SEM (Scanning Electronic Microscope), one can observe that the crystal phase in the exterior portion are different from those of the interior portion, as best shown in FIG. 3.

FIG. 4 shows a cross section view of crystal structure of the hemihydrate calcium sulfate tablet prior to relative humidity treatment, wherein the cross section is magnified 3000 times under SEM (Scanning Electronic Microscope).

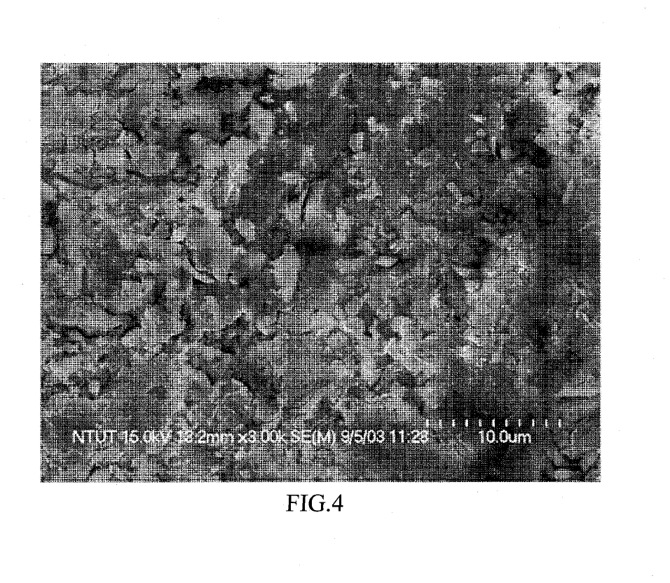

FIG. 5 shows a cross section view of crystal structure of the hemihydrate calcium sulfate tablet after relative humidity treatment, wherein the cross section is magnified 1500 times under SEM (Scanning Electronic Microscope) to illustrate an exterior portion.

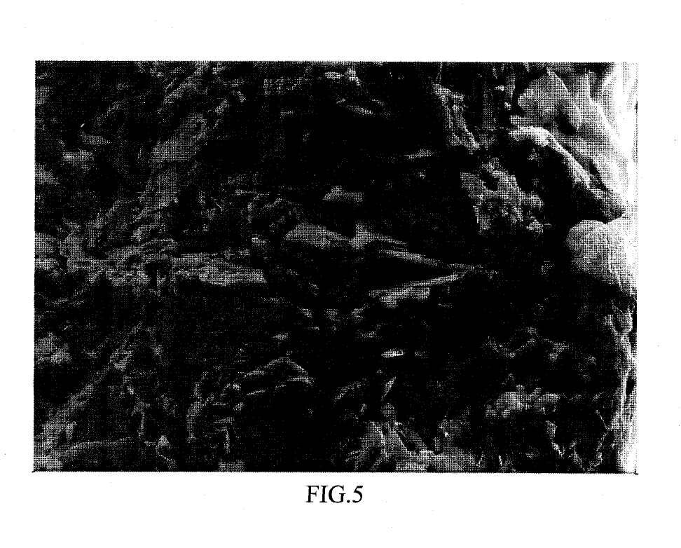

FIG. 6 shows a cross section view of crystal structure of the hemihydrate calcium sulfate tablet after relative humidity treatment, wherein the cross section is magnified 1500 times under SEM (Scanning Electronic Microscope) to illustrate an interior portion.

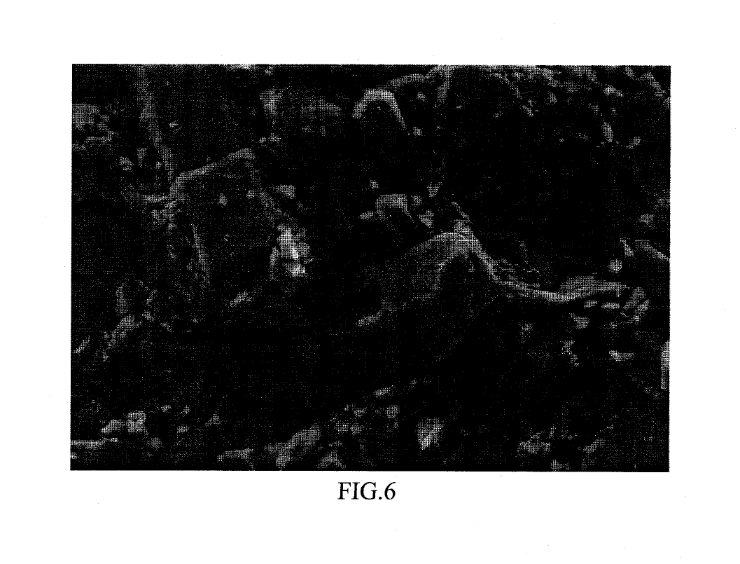

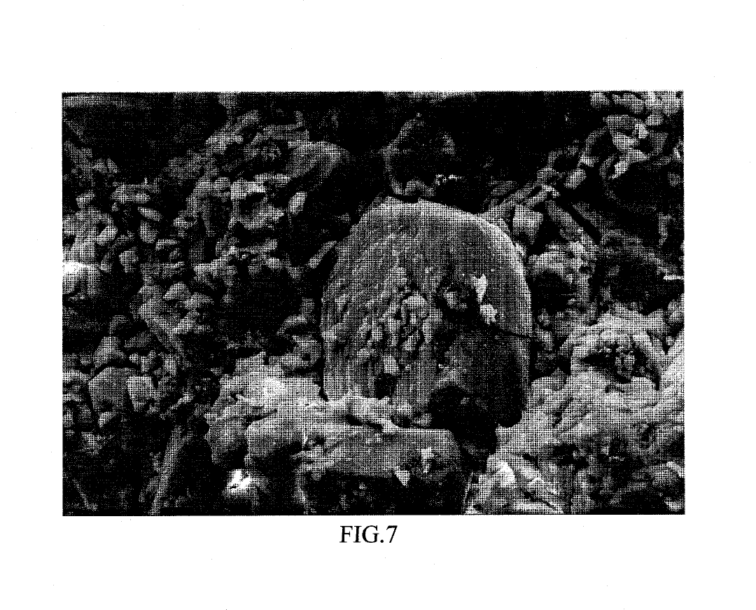

FIG. 7 shows a cross section view of crystal structure of the hemihydrate calcium sulfate tablet after relative humidity treatment, wherein the cross section is magnified 1500 times under SEM (Scanning Electronic Microscope) to illustrate an intermediate portion.

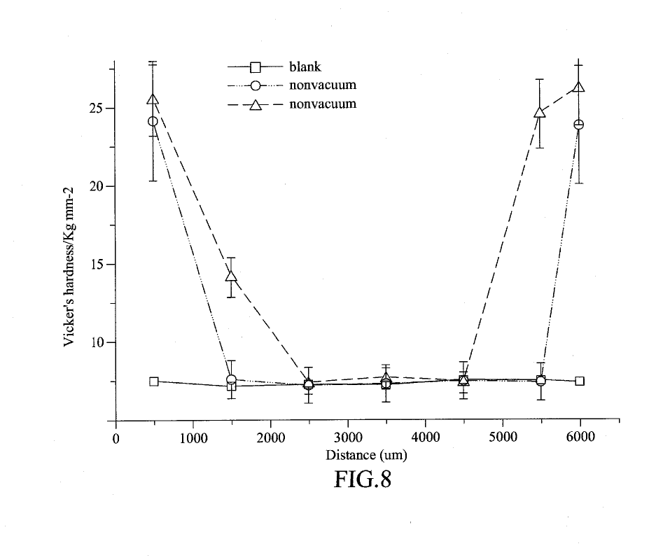

FIG. 8 shows a graph of representing hardness test of the hemihydrate calcium sulfate tablet carried out by Vicker's Hardness Tester, wherein L1 denotes the hardness distribution of the hemihydrate calcium sulfate tablet which has first gone through the vacuum suction process while L2 denotes the hardness distribution of the hemihydrate calcium sulfate tablet which has not gone through the vacuum suction process.

From the above-mentioned Vicker's test, one can observe that if the moist hemihydrate calcium sulfate tablet is passed through a heat treatment for 12 hours under the constant temperature of 40° C. with relative humidity 95% RH, the hardness of the moist hemihydrate calcium sulfate tablet is increased and the increase amount is due to the vacuum suction process.

In the second embodiment, the compact hemihydrate calcium sulfate tablet of 3 mm×5 mm is prepared under the relative humidity 95% RH, the heating time 12 hrs, the constant temperature 40° C. and the vacuum suction process (40° C., 1 hr), after which, an amount of 100 gm is taken for disposing into a grinder (60 rpm) for forming particles of hemihydrate calcium sulfate. The particles are passed through a 120 mesh sieve to obtain a plurality of slag, which pass through the sieve, and a plurality of granule, which are left over in the sieve. When each granule is viewed under SEM (Scanning Electronic Microscope), the granule has a diameter ranging 200 μm ˜1500 μm while the slag has a diameter ranging 50 μm ˜200 μm.

The plurality of granule and slag obtained by the above-mentioned method are mixed together with another amount of particles of hemihydrate calcium sulfate at a predetermined particle ratio and powders/water ratio to form a hemihydrate calcium sulfate composite material, which is later, mixed together with a solution to form a slurry of bone cement. The slurry of bone cement is left for solidification. During the solidification process, a compression strength of the slurry of bone cement is measured for several times. The measured compression strength is compared relative to a blank compression strength, wherein the blank compression strength is measured after solidification of the bone cement at the optimum water ratio. In case the measured compression strength is not compatible with the blank compression strength, the measured compression strength is adjusted until it is compatible with the blank compression strength.

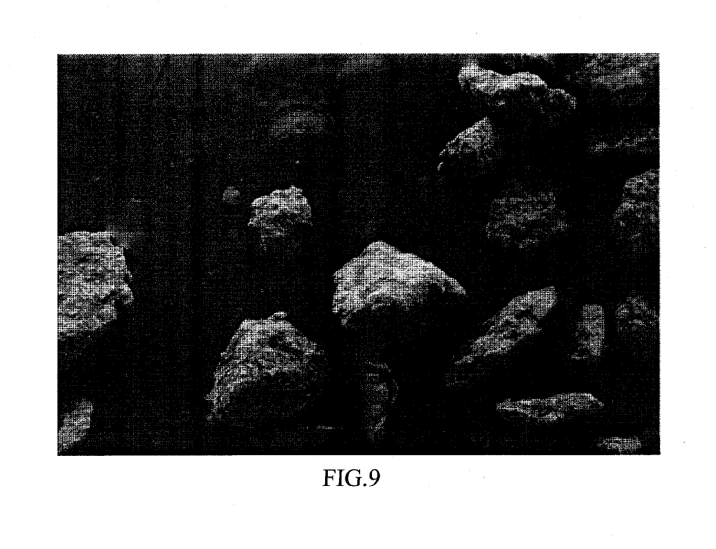

FIG. 9 illustrates a distribution state of a plurality of slag viewed and magnified 50 times under SEM (Scanning Electronic Microscope).

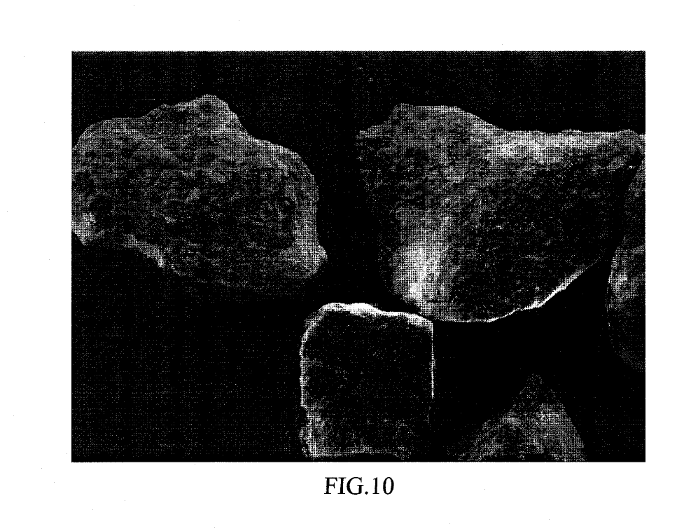

FIG. 10 illustrates a distribution state of a plurality of granule viewed and magnified 50 times under SEM (Scanning Electronic Microscope).

In the third embodiment, the plurality of granule and slag obtained as stated in the second embodiment are mixed together with another amount of particles of hemihydrate calcium sulfate at a predetermined particle ratio (in weight) as shown by the following table.

| | No. | | | | | | | | |
|---|---|---|---|---|---|---|---|---|---|
| | 1 | 2 | 3 | 4 | 5 | 6 | 7 | 8 | 9 |
| Calcium | 3.75 | 3.75 | 3.75 | 3 | 3 | 3 | 4.125 | 4.125 | 4.125 |
| sulfate | | | | | | | | | |
| hemihydrate | | | | | | | | | |
| (g) | | | | | | | | | |
| Granule | 3.375 | 0.375 | 1.875 | 4.125 | 0.375 | 2.25 | 3 | 0.375 | 1.875 |
| (g) | | | | | | | | | |
| Slag(g) | 0.375 | 3.375 | 1.875 | 0.375 | 4.125 | 2.25 | 0.375 | 3 | 1.5 |
| Water(c.c) | 3 | 3 | 3 | 3 | 3 | 3 | 3 | 3 | 3 |
| Compress | 12.71 | 10.05 | 11.48 | 10.87 | 7.69 | 8.45 | 18.73 | 12.82 | 13.51 |
| Strength(Mpa) | | | | | | | | | |
| Initial | 14 | 16 | 15 | 15 | 18 | 16 | 11 | 13 | 12 |
| setting | | | | | | | | | |
| time(min) | | | | | | | | | |
| Final | 62 | 66 | 64 | 65 | 69 | 66 | 55 | 60 | 57 |
| setting | | | | | | | | | |
| time(min) | | | | | | | | | |
| Cs:g:s | 5:4.5:0.5 | 5:0.5:4.5 | 5:2.5:2.5 | 4:5.5:0.5 | 4:0.5:5.5 | 4:3:3 | 5.5:4:0.5 | 5.5:0.5:4 | 5.5:2.5:2 |
| ratio | | | | | | | | | |

After mixing the composite consist according to the particle ratio shown in the above table, 7.5 gm of the composite consist from each group is mixed with 3 cc of de-ionized water for 60 seconds. A Vicat Needle is used to test the samples thus obtained to find out its initial setting time and final setting time. The samples are dried out for 36 hours and later a test of compression strength is carried out. As illustrated in column 7 of the table, the biomedical bone filler having the maximum compression strength is obtained by mixing under a ratio: 4.125 gm of hemihydrate calcium sulfate+3 gm of granule+0.375 gm of slag+3 cc of dilled water and after undergoing above-stated method.

In the fourth embodiment, the hemihydrate calcium sulfate 4.125 gm+3 gm of granule+0.375 gm of slag in column 7 of the third embodiment is mixed with a different volume of dilled water. An E.I.S (electrochemical impedance spectrum) is conducted to obtain the optimum water ratio. The AC Impedance test generally includes Frequency Response Analyzer S.I 1255 and Model EG&G 273A Potentiostat/Galvanostat, wherein Galvanostat generates 10 mV voltage for amplitude of AC signal while Frequency Response Analyzer provides a frequency range ranging 10¹˜10⁺⁵ Hz. It is to test the reflection effect from the external surface of the samples. In the third embodiment, the composite consist composed in the ratio amount shown in column 7 is to mix and blend with the water amount shown in the following table for 60 seconds and is poured into an acrylic mold having length of 50 mm, wide 10 mm and 40 mm height, where a standard Tri-Electrode Scanning is conducted under the testing temperature of 25° C. By using a blank sample to test its initial setting time for serving the purpose of testing a compression strength of the blank sample. Note that the compression strength is measured after solidification of the bone cement at the optimum water ratio. It is noted that the blank compression strength |z| is 1.77×10⁶. The composite consists are mixed with water according to the water ratio shown in the following table for serving as working electrodes, while foil, sheets and foil strings serve as guarding electrodes, Ag electrode serving as reference. Long term scanning operation is carried out to measure AC impedance values in order to achieve BODE table including frequency VS. total compression strength.

NO.

| | 1 | 2 | 3 | 4 |
|---|---|---|---|---|
| Water (c.c): L | 3 | 2.25 | 3.75 | 1.5 |
| Powder (g): P | 7.5 | 7.5 | 7.5 | 7.5 |
| P/L ratio | 2.5 | 3.33 | 2 | 5 |

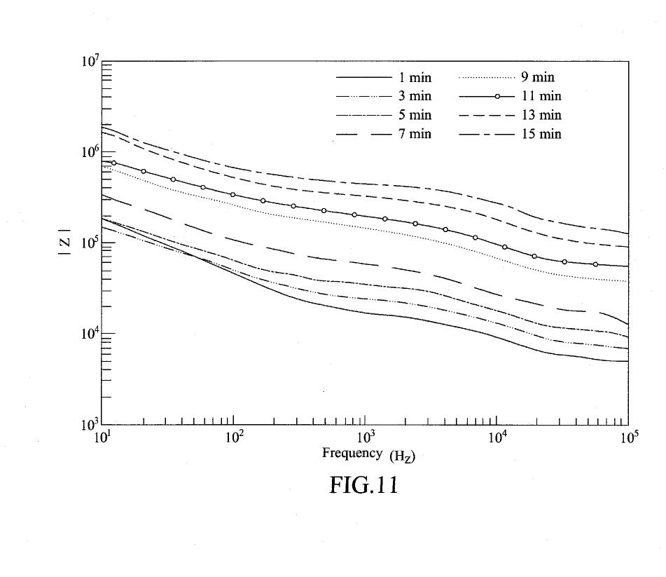

FIG. 11 is a graph based on power and water ratio 2.5 P/L of column 1 after conducting E. I. S (electrochemical impedance spectrum). As can be seen in the graph, when, the initial setting time is 11 minutes, the tested compression strength |z| is near 9.57×10⁵.

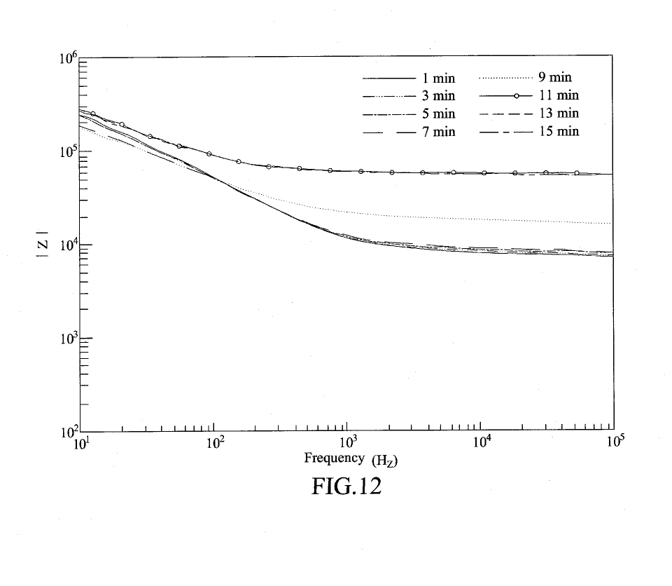

FIG. 12 is a graph based on power and water ratio 3.33 P/L of column 2 after conducting E. I. S (electrochemical impedance spectrum). As can be seen in the graph, after 11 minutes, the tested compression strength |z| is near 1.93×10⁵, which is quite a distance from the initial setting value.

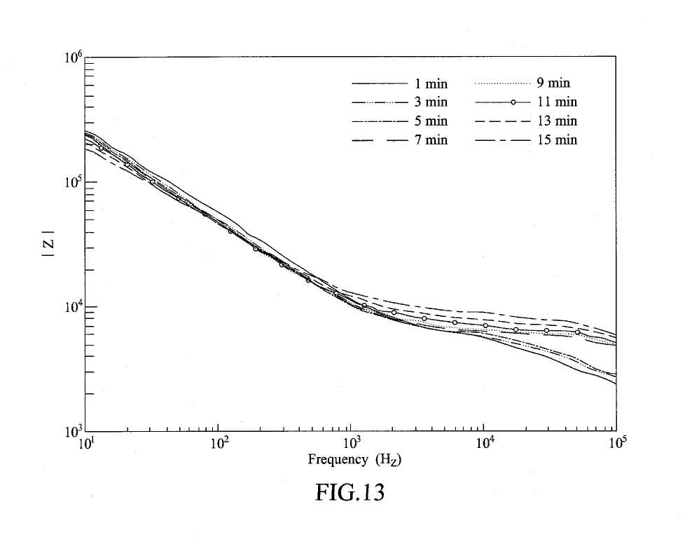

FIG. 13 is a graph based on power and water ratio 2 P/L of column 3 after conducting E. I. S (electrochemical impedance spectrum). As can be seen in the graph, after 11 minutes, the tested compression strength |z| is near 2.91×10⁵, which is quite a distance from the initial setting value.

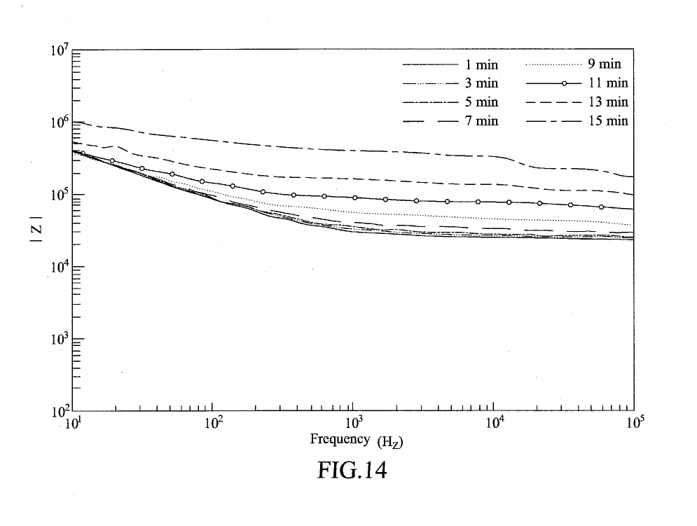

FIG. 14 is a graph based on power and water ratio 5 P/L of column 4 after conducting E. I. S (electrochemical impedance spectrum). As can be seen in the graph, after 11 minutes, the tested compression strength |z| is near 5.1×10⁵, which is quite a distance from the initial setting value.

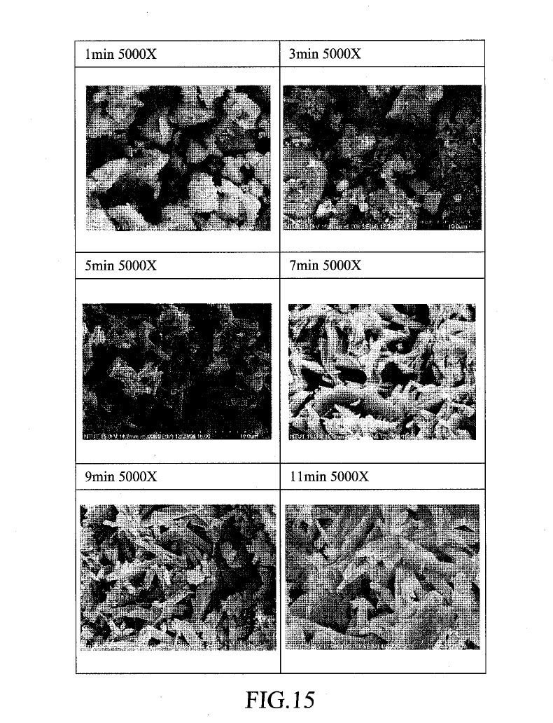

FIG. 15 shows the crystals of hemihydrate calcium sulfate magnified 5000 times under SEM

(Scanning Electronic Microscope) after solidification of the slurry of bone cement 1, 3, 5, 7, 9 and 11 minutes respectively.

By comparing the compression strengths of E. I. S relative to the BODE table, it is found that when P/L ratio=2.5, the measured compression strength |z| is near 9.57×10⁵ and similar to the initial setting time of the blank compression strength and is compatible to the initial setting time of the conventional Vicat Needle Test. In other words, one can predict the initial setting times of the hemihydrate calcium sulfate by conducting AC impedance analysis so as to determine the optimum water ratio. However, the conventional Vicat Needle Test for determining the initial setting times of the hemihydrate calcium sulfate fails to show the response of the initial setting stage so that the testing person may mistakenly insert the Vicat Needle at undesired times, thereby causing errors in measuring the compression strength.

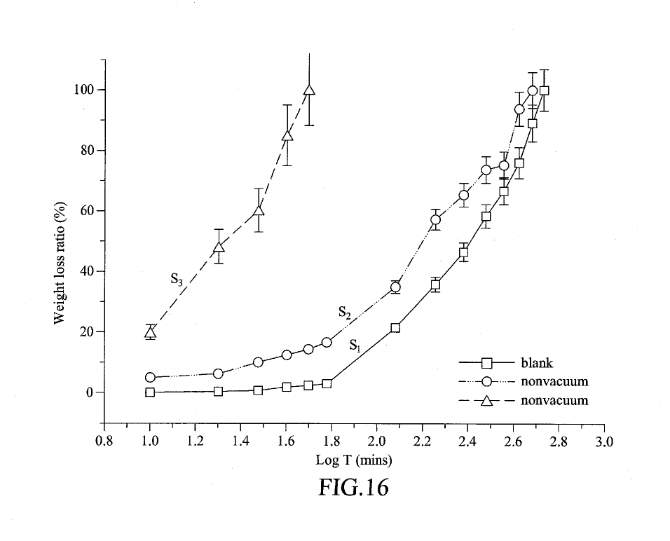

FIG. 16 shows the sixth embodiment of the biomedical bone filler of the present invention, wherein the particle ratio: hemihydrate calcium sulfate 4.125 gm:3 gm of granule:0.375 gm of slag in column 7 of the third embodiment is dried out (i) via the vacuum suction and (ii) without the vacuum suction. An equivalent amount of hemihydrate calcium sulfate is fetched for serving as the blank reference. Afterward, 3 cc of water is added into each group, is stirred for 60 seconds and is poured into an acrylic mold having length of 50 mm, wide 10 mm and 40 mm height. After 36 hours passed, the composite material is removed from the mold and is immersed within 100 ml of dilled water, wherein a solution is resulted due to dissolving action of the composite material. At every 24 hour interval, a predetermined amount of the solution is removed and is replaced with the same amount of dilled water, and relative weights of the materials are measured in order to understand their degradation. As can be seen in the drawing, line S1 (84 days to dissolve in water) represents the composite material that is dried out via the vacuum suction while line S2 (65 days to dissolve in water) represents the composite material that is dried out without the vacuum suction. Note that lines S1 and S2 respectively have larger degradation ability far greater than the blank compression strength line S3 (37 days to dissolve in water). From the above-mentioned experiment, it is noted that the biomedical bone filler manufactured according the present invention has concrete characteristic and feature.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A method for manufacturing biomedical bone filler comprising the steps of:
(j) preparing one biomedical hemihydrate calcium sulfate tablet;
(k) keeping said hemihydrate calcium sulfate tablet under a conditioning environment having constant temperature and humidity;
(l) controlling temperature of said conditioning environment at 40° C. with humidity ranging 50˜95% RH for 12 hours in order to form a moist hemihydrate calcium sulfate tablet, wherein said moist hemihydrate calcium sulfate tablet having an exterior portion consisting of dihydrate calcium sulfate crystal phase;
(m) drying out said moist hemihydrate calcium sulfate tablet under a vacuum condition in order to obtain a compact hemihydrate calcium sulfate tablet;
(n) cracking said compact hemihydrate calcium sulfate tablet into granules and slag;
(o) mixing particles of hemihydrate calcium sulfate with said granules and slag at a predetermined particle ratio and powders/water ratio to form a hemihydrate calcium sulfate composite material, which is mixed together with a solution to form a slurry of b one cement;
(p) carrying out a hardening process on said slurry of bone cement and measuring a compression strength of said slurry bone cement during said hardening process;
(q) comparing said measured compression strength relative to a blank compression strength, wherein said blank compression strength is measured after solidification of said bone cement at the optimum water ratio; and
(r) adjusting the optimum water ratio when said measured compression strength is not compatible with said blank compression strength until said measured compression strength is compatible with said blank compression strength.

2. The method according to claim 1, wherein said solution of the step (o) consisting of pure water or de-ionized water (DI water).

3. The method according to claim 1, wherein drying out said moist hemihydrate calcium sulfate tablet under said vacuum condition is carried out at least for 1 hour.

4. The method according to claim 1, wherein said moist hemihydrate calcium sulfate tablet of step (l) further includes an interior portion consisting of hemihydrate calcium sulfate crystals.

5. The method according to claim 1, wherein said slurry of bone cement of step (o) is composed of α and β phases of hemihydrate calcium sulfate salt.

6. The method according to claim 1, wherein each slag obtained by cracking said compact hemihydrate calcium sulfate tablet has a diameter smaller than 200 μm.

7. The method according to claim 1, wherein said granule obtained by cracking said compact hemihydrate calcium sulfate tablet has a diameter ranging 200˜1500 μm.

8. The method according to claim 1, wherein the biomedical bone filler having concrete characteristic is formed once a plurality amount of said granule and slag are mixed into the slurry of bone cement.

9. The method according to claim 1, wherein the biomedical bone filler has a ratio of hemihydrate calcium sulfate:granule:slag=5.5:4.0:0.5.

10. The method according to claim 1, wherein 2˜5 preferred amount of water is needed for mixing a plurality amount of granule and slag of dihydrate calcium sulfate into said slurry of bone cement to form the biomedical bone filler.

11. The method according to claim 10, wherein an AC Impedance analysis is implemented for obtaining said 2˜5 preferred amount of water.
